(19) United States
(12) Patent Application Publication
(10) Pub. No.: US 2008/0293772 A1
(43) Pub. Date: Nov. 27, 2008

(54) SALTS OF PICOTAMIDE

(76) Inventors: Herman J. Kempen, Dornach (CH); Barbara Staehelin, Binningen (CH)

Correspondence Address: WENDEROTH, LIND & PONACK, L.L.P., 2033 K STREET N. W., SUITE 800, WASHINGTON, DC, 20006-1021, US

(21) Appl. No.: 12/124,790

(22) Filed: May 21, 2008

(30) Foreign Application Priority Data
May 21, 2007 (EP) 07108522.9

(51) Int. Cl. A61K 31/4545; C07D 401/06; A61P 9/00; A61P 3/10; A61P 1/00

(52) U.S. Cl. 514/332; 546/262

(57) ABSTRACT

The invention relates to salts of picotamide (N,N′-bis-(3-picolyl)-4-methoxyisophthalamide) with strong acids, to pharmaceutical compositions containing picotamide salts, and to a method of treatment of cardiovascular and related diseases using picotamide salts. Particularly useful are picotamide hydrochloride and picotamide mesylate, preferably on a carbohydrate carrier such as hydroxymethylpropylcellulose.

### FIELD OF THE INVENTION

The invention relates to salts of picotamide (N,N′-bis-(3-picolyl)-4-methoxyisophthalamide), a process of preparation thereof, pharmaceutical compositions containing these, and use of these salts in the treatment of diseases.

### BACKGROUND OF THE INVENTION

It was described already 30 years ago that N,N′-bis-(3-picolyl)-4-methoxyisophthalamide, hereinafter referred to by its international non-proprietary name “picotamide”, is a compound having a high fibrinolytic and anticoagulant activity (French Patent 2 100 850) as well as a good platelet antiaggregant activity (U.S. Pat. No. 3,973,026). It is now known that picotamide is a dual acting thromboxane A2 (TXA2) antagonist and thromboxane synthase inhibitor, and an inhibitor of platelet aggregation and vascular constriction (see e.g. Gresele et al., Thromb. Haemost. 61:479-84, 1989; Cattaneo et al., Thromb. Res. 62:717-24, 1991). In patients with peripheral artery disease, administration of this agent leads to a reduced risk to suffer from fatal and non-fatal vascular events (Balsano et al., Circulation 87:1563-1569, 1993) and unstable angina (Neri Serneri et al., Coron. Artery Dis. 5:137-145, 1995). In patients with peripheral artery disease and diabetes, picotamide was shown to reduce cardiovascular mortality (Neri Serneri, Eur. Heart J. 25:1845-52, 2004).

The anhydrous form of picotamide as described in French Patent 2 100 850 has a melting point of 124° C. A crystal form of picotamide monohydrate has been described in UK patent 2 080 288 as having a melting point of 95-97° C. As described in this UK patent, after oral administration of the monohydrate the time needed to reach the maximum inhibitory effect in man was 4 hours, whereas after administration of anhydrous picotamide maximum activity is reached only after 8 hours. Further, according to this patent the anhydrous form is relatively unstable and difficult to use in pharmaceutical formulations. The crystalline monohydrate is more stable and easier to use for pharmaceutical purposes. However, the time to reach maximum activity is still relatively long suggesting that the bioavailabilty of the compound is not yet optimal.

The tartrate salt of picotamide was used by M. Berrettini et al., Eur J Clin Pharmacol 39:495-500, 1990 for in vitro tests in aqueous solution. For in vivo tests, the authors used the commercially available product sold under the trade name Plactidil™ containing the free base N,N′-bis-(3-picolyl)-4-methoxyisophthalamide.

### SUMMARY OF THE INVENTION

The invention relates to salts of picotamide (N,N′-bis-(3-picolyl)-4-methoxyisophthalamide) with strong acids, in particular to the hydrochloride and the mesylate salt, to a process of preparation thereof, to pharmaceutical compositions containing picotamide salts, preferably in admixture with a carbohydrate carrier, to the use of picotamide salts for the manufacture of pharmaceutical compositions for the treatment of cardiovascular and related diseases, and to a method of treatment of cardiovascular and related diseases using picotamide salts.

### BRIEF DESCRIPTION OF THE FIGURES

FIG. 1. Raman spectrum of picotamide hydrochloride on hydroymethylpropylcellulose. X-axis: Wave number [cm⁻¹], Y-axis: Raman emission.

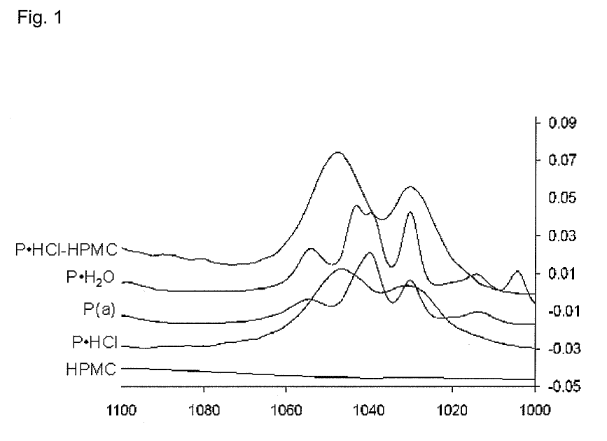

Bottom curve: Hydroxymethylpropylcellulose (HMPC). Second curve from below: Picotamide hydrochloride (P.HCl). Third curve from below: Picotamide anhydride (P(a)). Forth curve from below: Picotamide monohydrate (P.H₂O). Top curve: Picotamide hydrochloride on HMPC, weight ratio polymer to picotamide 2:1 (P.HCl-HMPC).

FIG. 2. Dissolution kinetics of picotamide monohydrate free base (-♦-), crushed Plactidil™ tablet (-▪-), and of picotamide hydrochloride (-▴-) or picotamide mesylate on hydroxymethylpropylcellulose (--).

X-axis: Time [min], Y-axis: Dissolution [%].

### DETAILED DESCRIPTION OF THE INVENTION

Picotamide is the international non-proprietary name for N,N′-bis-(3-picolyl)-4-methoxyisophthalamide of the formula

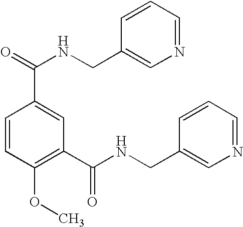

Although carboxamide functions contain a nitrogen atom, these functions are not basic enough to form acid addition salts. Pyridinium salts are known, but acid addition salts are rarely used in practice because of the low basicity of the pyridine nitrogen atom. A salt of picotamide with tartaric acid has been reported in the literature (M. Berrettini et al., Eur J Clin Pharmacol 39: 495-500, 1990), but solid acid addition salts of picotamide have not been used in clinical or pharmaceutical practice. It has now surprisingly been found that picotamide acid addition salts with strong acids can be readily formed, can be stabilized in solid form in the presence of carbohydrate polymers, have useful properties in the formation of pharmaceutical formulations, and show advantageous dissolution behaviour of relevance for the application as a medicament.

Acid addition salts with strong acids may be formed from inorganic or organic acids. Strong acid in the sense of the present invention are acids with a pKₐ value below 2.5, preferably below 2.0, in particular below 1.5. Suitable inorganic acids are, for example, halogen acids, such as hydrochloric or hydrobromic acid, sulfuric acid, or phosphoric acid. Suitable organic acids are, for example, carboxylic, phosphonic, sulfonic or sulfamic acids, for example oxalic acid, maleic acid, amino acids, such as glutamic acid or aspartic acid, methylmaleic acid, dimethylmaleic (citraconic) acid, difluoracetic acid, trifluoroacetic acid, methane- or ethane-sulfonic acid, 2-hydroxyethanesulfonic acid, ethane-1,2-disulfonic acid, 10-camphorsulphonic acid, benzenesulfonic acid, 2-naphthalenesulfonic acid, 1,5-naphthalene-disulfonic acid, o-, m- or p-toluenesulfonic acid, methylsulfuric acid, ethylsulfuric acid, dodecylsulfuric acid, N-cyclohexylsulfamic acid, or N-methyl-, N-ethyl- or N-propyl-sulfamic acid.

Particularly useful are salts from pharmaceutically acceptable acids, but for isolation or purification purposes it is also possible to use pharmaceutically unacceptable salts, for example salts with picric or with perchloric acid.

Preferred picotamide salts are the hydrochloride, hydrobromide, sulphate, phosphate, oxalate, maleate, trifluoroacetate, mesylate (methanesulphonate), p-toluenesulphonate, or 10-camphorsulphonate.

Most preferred are picotamide salts formed with hydrochloric acid (i.e. the hydrochloride) or with sulfonic acids, e.g. with methanesulfonic acid (i.e. the mesylate).

The advantage of these salts (for example obtained in complex with carbohydrate polymers) over the free base of picotamide (anhydrous or as the monohydrate) consists of their more rapid and complete dissolution in neutral aqueous environment, which is important when used in oral pharmaceutical formulations. The mentioned picotamide salts readily dissolve in neutral or slightly basic environment of the mouth and the small intestinal lumen, thereby becoming more rapidly available for absorption in these compartments by the body and so exerting its beneficial effects more rapidly and completely than provided for by the current pharmaceutical formulation containing the free base form.

Picotamide, and especially picotamide salts as described herein, are effective inhibitors of platelet aggregation and vascular constriction, improve the walking distance of patients with peripheral artery disease, are effective in secondary prevention of transient ischemic attacks and stroke (superior to aspirin), are effective in secondary prevention of cardiovascular events in diabetic patients with peripheral artery disease or carotid atherosclerosis, are effective in reducing anginal events in patients with unstable angina or effort angina (where aspirin has no effect), are effective in reducing albuminuria in patients with micro-albuminuria, reduce progression of plaques in carotid atherosclerosis, reduce aura in migraine patients, reduce serum creatinine and pulmonary pressure in congestive heart failure patients, and are useful in related cardiovascular problems.

Picotamide salts according to the invention can therefore be used in the treatment of cardiovascular diseases. For example, picotamide salts can be used for the prevention of morbidity and mortality due to acute coronary syndrome, myocardial infarction, transient ischemic attacks, stroke and leg infarction, in patients who are at risk for these diseases, and/or in patients who already have suffered such incidents. These effects are the result of the ability of picotamide and picotamide salts to prevent arterial thrombosis by inhibiting platelet aggregation and to enhance fibrinolysis, thus reducing the occurrence of occlusive clots in coronary, carotid and leg arteries. The advantage of picotamide salts over aspirin as anti-platelet agent is that picotamide salts do not lead to erosions and ulcerations of the stomach epithelium.

Picotamide salts can further be used for the symptomatic treatment of angina pectoris and intermittent claudication. These effects are the result of ability of picotamide and picotamide salts to induce arterial vasorelaxation.

Picotamide salts can further be used for prevention of end-stage renal disease in patients with diabetic nephropathy, for the treatment and prevention of diabetic neuropathy and retinopathy, for the reduction of migraine and of aura in migraine patients, and for the reduction of pulmonary blood pressure in patients with chronic heart failure.

Picotamide salts are likewise useful in treatment of premature labour and of asthma, both based on the ability of picotamide and picotamide salts to induce relaxation of smooth muscle cells, and in the treatment of inflammatory bowel disease.

The present invention relates also to pharmaceutical compositions that comprise a picotamide salt as active ingredient and that can be used especially in the treatment of the diseases mentioned hereinbefore. Compositions for enteral administration, such as nasal, buccal, rectal or, especially, oral administration, are preferred. The compositions comprise the active ingredient alone or, preferably, together with a pharmaceutically acceptable carrier. The dosage of the active ingredient depends upon the disease to be treated and upon the species, its age, weight, and individual condition, the individual pharmacokinetic data, and the mode of administration.

The present invention relates especially to pharmaceutical compositions that comprise a picotamide salt on a solid pharmaceutically acceptable carrier, preferably a solid carrier composed of carbohydrate units. Such carriers are, for example, sugars, such as mannose, lactose, fructose, glucose, sucrose or saccharose, sugar alcohols, such as mannitol, xylitol or sorbitol, starches, for example corn, wheat, rice or potato starch, cellulose preparations, for example microcrystalline cellulose, methylcellulose, hydroxypropylcellulose, hydroxypropyl methylcellulose, or sodium carboxymethylcellulose, guar gum, carrageenan, or acacia gum.

Preferred carriers are microcrystalline cellulose, and in particular hydroxymethylpropylcellulose (HMPC) and sodium carboxymethylcellulose.

The invention relates also to pharmaceutical compositions comprising picotamide salts for use in a method for the prophylactic or especially therapeutic management of the human or animal body, in particular in a method of treating cardiovascular and related diseases mentioned above.

The invention relates also to processes for the preparation of pharmaceutical compositions comprising picotamide salts.

The pharmaceutical compositions comprise from approximately 1% to approximately 95% picotamide salts, preferably between 20% and 50% picotamide salts. Unit dose forms are, for example, tablets, mini-tablets, granules, capsules containing mini-tablets or granules, lozenges or chewing-gums.

The pharmaceutical compositions of the present invention are prepared in a manner known per se, for example by means of conventional mixing, granulating, dissolving or lyophilizing processes.

Pharmaceutical compositions for oral administration can be obtained, for example, by mixing picotamide (free base) with a solution of the acid for salt formation and with one or more solid carriers, if desired or necessary, by the inclusion of additional excipients, evaporating the solvent and, if desired granulating or tabletting the resulting mixture and optionally filling granules or mini-tablets into capsules, or adding the mixture to a suitable material for the preparation of lozenges or chewing-gums.

Suitable additional carriers are especially fillers, such as the sugars, sugar alcohols and cellulose preparations mentioned above as carriers, and phosphates and silicates, and also binders, such as starches, for example corn, wheat, rice or potato starch, methylcellulose, hydroxypropyl methylcellulose, sodium carboxymethylcellulose or polyvinylpyrrolidone, and/or disintegrators, such as the mentioned starches, also carboxymethyl starch, crosslinked polyvinylpyrrolidone, alginic acid or a salt thereof, such as sodium alginate. Additional excipients are especially flow conditioners and lubricants, for example silicic acid, talc, stearic acid or salts thereof, such as magnesium or calcium stearate, and/or polyethylene glycol, or derivatives thereof.

Dyes or pigments may be added to the tablets, granules, lozenges or chewing-gums, for example for identification purposes or to indicate different doses of picotamide salts.

Pharmaceutical compositions for oral administration also include hard capsules consisting of gelatin, and also soft, sealed capsules consisting of gelatin and a plasticizer, such as glycerol or sorbitol. The capsules may contain the picotamide salt in the form of granules, for example in admixture with fillers, such as corn starch, binders, and/or glidants, such as talc or magnesium stearate, and optionally stabilizers.

Pharmaceutical compositions suitable for rectal administration are, for example, suppositories that consist of a combination of the picotamide salt and a suppository base. Suitable suppository bases are, for example, natural or synthetic triglycerides, paraffin hydrocarbons, polyethylene glycols or higher alkanols.

Pharmaceutical compositions of the invention comprising a picotamide salt as described hereinbefore are more convenient than the prior art compositions containing anhydrous picotamide or crystalline picotamide monohydrate. Picotamide salts dissolve very rapidly and more completely in neutral to slightly basic environment. Dissolution therefore already can occur in the mouth with saliva, allowing buccal absorption, with a rapid onset of the desired activity of the active ingredient. This is particularly important, if patients with angina attacks or claudication pain want relief as quickly as possible. Buccal absorption is also observed in pharmaceutical compositions in the form of lozenges containing soft gum. Such lozenges may be particularly important for patients having difficulties in chewing and/or swallowing tablets.

It has been observed that picotamide salts on a carbohydrate carrier, especially picotamide salts on HPMC carrier, are free flowing amorphous powders and very convenient for processing into desired dosage forms, e.g. tablets. They are also particularly useful as starting materials for prolonged release formulations.

The present invention relates furthermore to a method for treatment of cardiovascular or related diseases which comprises administering a picotamide salt in a quantity effective against said disease, to a warm-blooded animal requiring such treatment. The picotamide salts can be administered as such or especially in the form of pharmaceutical compositions, prophylactically or therapeutically, preferably in an amount effective against the said diseases, to a warm-blooded animal, for example a human, requiring such treatment. In the case of an individual having a bodyweight of about 70 kg the daily dose administered is from approximately 0.05 g to approximately 5 g, preferably from approximately 0.25 g to approximately 1.5 g, of a picotamide salt.

The present invention relates also to the use of a picotamide salt, especially those mentioned as being preferred, as such or in the form of a pharmaceutical formulation with at least one pharmaceutically acceptable carrier for the therapeutic and also prophylactic management of one or more of the diseases mentioned hereinabove, in particular cardiovascular diseases. The invention furthermore relates to the use of a picotamide salt, especially those mentioned as being preferred, for the manufacture of pharmaceutical compositions for the treatment of cardiovascular and related diseases.

The preferred dose quantity, composition, and preparation of pharmaceutical formulations (medicines) which are to be used in each case are described above.

The following Examples serve to illustrate the invention without limiting the invention in its scope.

### EXAMPLES

### Example 1

### Salt Formation and Salt Stability

### Salt Formation

500 ml (1 equivalent) or 1000 ml (2 equivalents) of a 0.05 M solution of the acid is added to 500 ml of a 0.05 M solution of picotamide monohydrate (European Pharmacopoeia BP 907-F67029) in tetrahydrofuran (THF), methanol or a mixture of isopropanol/THF. If a spontaneous crystallization occurs after the salt formation, the solid is isolated by filtration. If precipitation does not occur, the solvent is slowly removed by evaporation at 25° C.

If not crystalline the solid remaining after evaporation is suspended in 500 ml of a suitable solvent for induction of crystal formation (e.g. acetone, water, diethyl ether, acetonitrile and mixtures thereof). The mixture is placed on a shaker for 24 h at 25° C. After the equilibration the solvent is slowly removed by evaporation.

### Raman Spectra

A Raman spectrum is acquired after additional vacuum drying at <0.01 mbar for 72 h. Raman measurements are performed with a Bruker RFS 100/S Raman spectrometer, excitation laser power 400 mW, resolution: 2 cm⁻¹.

### Salt Formation in Presence of Excipients

Picotamide monohydrate (40 mg/ml) is suspended in water and 1 or 2 equivalents of either hydrochloric acid or methanesulfonic acid (in 1 M concentration) are added to give a clear solution. To each part of picotamide monohydrate, two parts (per weight) of either mannitol or acacia gum is added. After complete dissolution of the excipient, the water is evaporated at 50° C. in a vacuum oven.

### HPLC Analysis

Column: Phenomenex Hypersil C18-BDS, 4.6×150 mm, 5 μm, flow 1 ml/min, 25° C., detection at 254 nm. Ammonium acetate buffer 10 mM, pH 9, containing 15% methanol. After 17 min, the percentage of methanol is increased to 80%. The sample is dissolved in methanol/water 1:1.

### Spectra Analysis

Potential candidates for salts are identified if the microscopic picture analysis shows evidence of a crystalline structure and/or the Raman spectrum is different compared to a combined spectrum of picotamide and the acid used. The Raman spectrum of the excipient is taken into account in the spectra analysis.

Picotamide Stability Under Acidic Conditions

Picotamide is dissolved in hydrochloric acid 1 M and stored at room temperature and 50° C. for three days. The picotamide content was assessed with HPLC and found to be 99.2% and 98.1%, respectively. These results show that formation of picotamide salts under acidic conditions does not give rise to significant picotamide degradation.

### Picotamide Salt Stability

Salts were formed in THF. Degradation is determined by HPLC: Acetate 0.6%, citrate 0.2%, fumarate 6.7%, hydrobromide 35%, hydrochloride 0.6%, hydrochloride (anhydrous) 0.0%, maleate 11.5%, mesylate 0.1%, oxalate 0.4%, sulfate 0.1%, tartrate 0.1%.

The picotamide content of the hydrochloride and mesylate salt coated on acacia gum were further tested with HPLC after three days at room temperature. The contents were found to be 98% and 97%, respectively.

On extensive drying at reduced pressure, salts with volatile acids tend to reform crystalline picotamide (free base), as judged from microscopic picture analysis.

### Example 2

### Picotamide Salts on Carbohydrate Carriers

Picotamide anhydrate (Sai Advantium Ltd.), an equimolar amount of hydrochloric acid (Merck) or methanesulfonic acid (Fluka, ≧99.0%), and one of the following water-soluble polymers were mixed in aqueous solution, evaporated and dried:
hydroxypropylmethylcellulose E5 (HPMC)
sodium carboxymethylcellulose (CMC)
hydroxypropylcellulose (HPC)
acacia gum (pharmaceutical grade, Colloide Naturels, France)
polyvinylpryrrolidone VA64 (PVP)

The weight ratio of polymer to picotamide salt was 0.1:1, 0.25:1, 1:1, 2:1, 3:1 and 5:1.

Mixtures with suitable flow properties were further tested for hygroscopicity at 25° C., 60% relative humidity and at 40° C., 75% relative humidity for a period of 6 weeks. During storage Raman spectra were acquired and water uptake was analysed by analysis of weight gain at predetermined time intervals.

The following results were obtained:

Picotamide hydrochloride and picotamide mesylate on HPMC and CMC had reasonable flow properties and only minimal hygroscopicity at weight ratios 1:1, 2:1, 3:1 and 5:1. The composition on HPMC did not increase in weight and remained solid after storage for 6 weeks at 40 degrees centigrade at 60% or 75% relative humidity.

Picotamide hydrochloride and picotamide mesylate on HPC and acacia gum had reasonable flow properties and acceptable hygroscopicity at weight ratio 2:1, 3:1 and 5:1 at 25° C., but lost these flow properties on storage at 40° C.

Picotamide hydrochloride and picotamide mesylate on PVP had reasonable flow properties to start with but lost these flow properties on storage due to hygroscopicity.

### Example 3

### Dissolution Measurements of Picotamide Salts on HPMC and Picotamide Free Base

### Manufacture of Picotamide Salt HPMC Complex 2:1

1 g picotamide anhydrate and an (equimolar) amount of HCl or methanesulfonic acid are dissolved in 50 ml of water. 2 g hydroxypropylmethylcellulose E5 (HPMC) are added and the mixture stirred until all HPMC is in solution. A first fraction of the water is evaporated by freeze-drying. The remaining water is removed by azeotropic distillation with toluene at 60° C. and 20 mbar. The obtained material is dried at 50° C. for 4 h and room temperature for 24 h at 200 mbar, then ground to a powder. The complexes had an off-white to beige colour and did not appear to be hygroscopic at ambient conditions.

### Dissolution Studies

155 mg of picotamide monohydrate, 500 mg of picotamide mesylate HPMC complex, 500 mg of picotamide chloride HPMC complex, and 192 mg Plactidil® granulate (containing 150 mg picotamide) prepared from tablets by crushing the tablets by means of a mortar and pestle, were added to a USP dissolution equipment containing 250 ml bicarbonate buffer (29 mM sodium bicarbonate adjusted to pH 7.7), which was kept at 37° C. and stirred at 50 rpm. At predetermined time intervals (1, 5, 10, 15, 30, 60 min) samples were drawn, filtered through a 0.45 μm pore size filter, and the picotamide content was determined by HPLC in the filtrate. After 60 minutes the pH was decreased to pH 1 by addition of 1 ml HCl 37% and stirred for another 30 minutes. The amount of picotamide found after filtration was regarded as the total amount of picotamide.

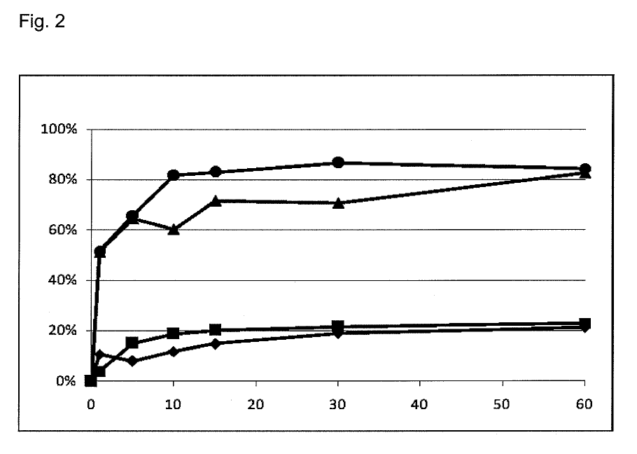

The results are shown in FIG. 2.

1. An acid addition salt of picotamide with a strong acid.

2. The picotamide salt according to claim 1, wherein the strong acid has a pKₐ value below 2.5.

3. The picotamide salt according to claim 1, wherein the strong acid has a pKₐ value below 2.

4. The picotamide salt according to claim 1, which is an acid addition salt of a halogen acid, sulfuric acid, phosphoric acid, carboxylic acid, phosphonic acid, sulfonic acid, or sulfamic acid.

5. The picotamide salt according to claim 1, which is picotamide hydrochloride, picotamide hydrobromide, picotamide sulphate, picotamide phosphate, picotamide oxalate, picotamide maleate, picotamide trifluoroacetate, picotamide mesylate, picotamide p-toluenesulphonate, or picotamide 10-camphorsulphonate.

6. The picotamide salt according to claim 1, which is picotamide hydrochloride.

7. The picotamide salt according to claim 1, which is picotamide mesylate.

8. A pharmaceutical composition comprising a picotamide salt according to claim 1.

9. A pharmaceutical composition according to claim 8 comprising a picotamide salt in admixture with a carbohydrate carrier.

10. A pharmaceutical composition according to claim 8 comprising a picotamide salt in admixture with hydroxymethylpropylcellulose.

11-12. (canceled)

13. A method of treatment and prevention of cardiovascular diseases, angina pectoris, intermittent claudication, diabetic nephropathy, migraine, pre-term labour, asthma, and inflammatory bowel disease, comprising administering to a patient in need thereof a therapeutically or prophylactically effective amount of a picotamide salt according to claim 1.
